(19) United States
(12) Patent Application Publication
(10) Pub. No.: US 2007/0135697 A1
(43) Pub. Date: Jun. 14, 2007

(54) Method and apparatus for providing sensor guard for data monitoring and detection systems

(75) Inventor: Christopher V. Reggiardo, Castro Valley, CA (US)

Correspondence Address: JACKSON & CO., LLP, 6114 LA SALLE AVENUE, SUITE 507, OAKLAND, CA, 94611-2802, US

(73) Assignee: TheraSense, Inc., Alameda, CA (US)

(21) Appl. No.: 10/986,542

(22) Filed: Nov. 10, 2004

Related U.S. Application Data
(60) Provisional application No. 60/563,369, filed on Apr. 19, 2004.

(51) Int. Cl. A61B 5/05

(52) U.S. Cl. 600/347

(57) ABSTRACT

Method and apparatus for providing sensor guard for data monitoring and detection system having a sensor for detecting one or more glucose levels, the sensor including a work electrode disposed on a base material, a reference electrode disposed on the base material, and a guard electrode disposed on the base material, where the guard electrode is maintained substantially at equipotential to the work electrode, and a transmitter operatively coupled to the work electrode and the reference electrode of the sensor for receiving said detected glucose levels, where the transmitter is further configured to transmit a respective signal corresponding to each of the detected glucose levels using a data transmission protocol including wireless data transmission protocols, to a receiver which is configured to receive the transmitted signals corresponding to said detected glucose levels is provided. The method and apparatus may also include insulin administration unit such as an insulin pump configured to be in data communication with the transmitter and/or the receiver for administering an appropriate insulin dosage based on the measured glucose levels.

### RELATED APPLICATION

This application claims priority under 35 USC §119 to Provisional Patent Application No. 60/563,369 filed on Apr. 19, 2004, entitled “Method And Apparatus for Providing Sensor Guard For Data Monitoring and Detection Systems”, the disclosure of which is incorporated herein by reference for all purposes.

### BACKGROUND

The present invention relates to data monitoring and detection systems. More specifically, the present invention relates to eletrometry detection systems and/or electro-physiology monitoring systems as used in radio frequency (RF) communication systems for data communication between portable electronic devices such as in continuous glucose monitoring systems.

Continuous glucose monitoring systems generally include a small, lightweight battery powered and microprocessor controlled system which is configured to detect signals proportional to the corresponding measured glucose levels using an electrometer, and RF signals to transmit the collected data. One aspect of such continuous glucose monitoring systems include a sensor configuration which is, for example, mounted on the skin of a subject whose glucose level is to be monitored. The sensor cell may use a three-electrode (work, reference and counter electrodes) configuration driven by a controlled potential (potentiostat) analog circuit connected through a simple contact system.

The current level detected by the work electrode of the sensor is relatively small such that even a small amount of leakage current from the reference or counter electrodes typically will affect the signal quality, and thus may have adverse effect upon the accuracy of the measured glucose level. This is especially true when foreign matter is present that causes a false high glucose reading that may lead to improper patient treatment. Furthermore, when the continuous glucose monitoring system is calibrated, the offset and gain of the sensor-transmitter pair is established. If the leakage current level changes (i.e., either increases or decreases), then the offset established will likely change and a resulting gain error may result for future calibration points.

To reduce the leakage current as much as possible and minimize the potential error in data reading, the reference electrode may be interposed between the work electrode and the counter electrode. This approach reduces the maximum potential from any of the reference or counter electrodes to the work electrode. However, even with such electrode configuration, the presence of foreign matter may cause a significant leakage current which could adversely affect patient care. In a two-electrode system without a reference electrode, the work electrode may be directly affected by leakage from the counter/reference electrode.

In view of the foregoing, it would be desirable to have a sensor configuration in data monitoring and detection systems such as in continuous glucose monitoring systems such that potential leakage current to work electrode in the sensor from the other electrodes is minimized.

### SUMMARY OF THE INVENTION

In view of the foregoing, in accordance with the various embodiments of the present invention, a separate guard contact (trace) may be provided in a multiple electrode sensor configuration in portable electronic devices such as in discrete or continuous glucose monitoring systems. The guard trace in one embodiment may be maintained at substantial equipotential to the work electrode, and provided to substantially physically encompass the work electrode so that current leakage path to the work electrode from any of the other electrodes (such as reference and/or counter electrodes) in the sensor configuration, may be protected by the guard contact.

Indeed, in accordance with one embodiment of the present invention, a guard contact may be disposed at equipotential to the work electrode to the sensor to reduce the possibility of a leakage current affecting the work electrode and eliminate the potential adverse results such as inaccurate data readings. This causes all leakage currents to be intercepted (captured) by the guard contact and the work electrode is thus unaffected even when foreign matter is present. The guard contact may be provided between the work electrode and the reference electrode in a three-electrode system, or between the work electrode, and reference/counter electrode in a two-electrode system.

In a further embodiment, the guard trace connected to the guard contact may be used to surround the work electrode and associated traces to reduce leakage to the greatest possible extent for a given sensor configuration. The guard trace may be extended from the system electronics through the contacts to the sensor to eliminate leakage currents resulting from contamination on the sensor. The extended guard contact and associated guard traces on the sensor in accordance with one embodiment is configured to substantially minimize the potential for leakage current to the work electrode in sensor configurations so as to substantially eliminate potential adverse results such as erroneous data reading.

Accordingly, potential error in the detected signals in the continuous glucose monitoring systems due to leakage current in the sensor of such systems may be minimized.

### BRIEF DESCRIPTION OF THE DRAWINGS

FIG. 1 illustrates a block diagram of a data monitoring and detection system for practicing one embodiment of the present invention;

FIG. 2 is a block diagram of the transmitter of the data monitoring and detection system shown in FIG. 1 in accordance with one embodiment of the present invention;

FIG. 3 illustrates the front end section of the analog interface of the transmitter in accordance with one embodiment of the present invention;

FIGS. 4A-4B respectively show detailed illustrations of the current to voltage circuit and the counter-reference servo circuit of the analog interface shown in FIG. 3 in accordance with one embodiment of the present invention;

FIGS. 5A-5B illustrate a top view and a side view of the two electrode sensor with guard trace in accordance with one embodiment of the present invention; and

FIGS. 6A-6B illustrate a top view and a side view of the three electrode sensor with guard trace in accordance with one embodiment of the present invention.

### DETAILED DESCRIPTION

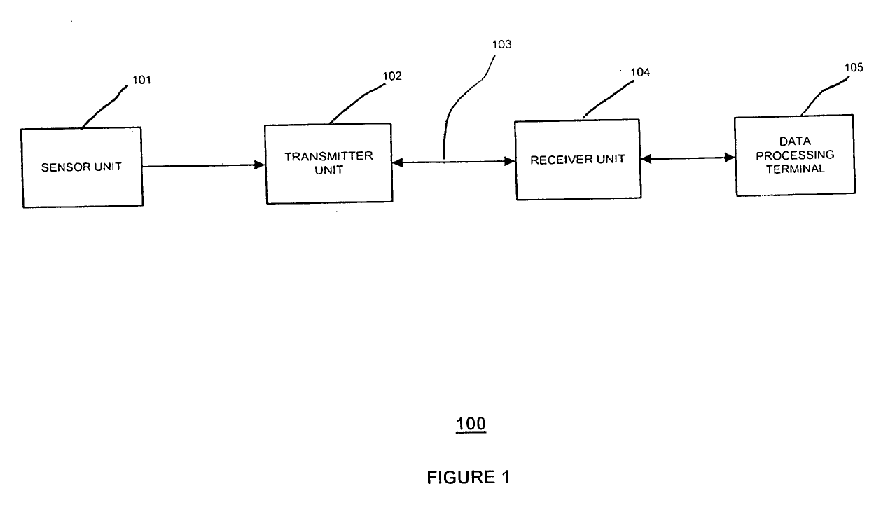

FIG. 1 illustrates a data monitoring and detection system such as, for example, a continuous glucose monitoring system 100 in accordance with one embodiment of the present invention. In such embodiment, the continuous glucose monitoring system 100 includes a sensor 101, a transmitter 102 coupled to the sensor 101, and a receiver 104 which is configured to communicate with the transmitter 102 via a communication link 103. The receiver 104 may be further configured to transmit data to a data processing terminal 105 for evaluating the data received by the receiver 104. Only one sensor 101, transmitter 102, communication link 103, receiver 104, and data processing terminal 105 are shown in the embodiment of the continuous glucose monitoring system 100 illustrated in FIG. 1. However, it will be appreciated by one of ordinary skill in the art that the continuous glucose monitoring system 100 may include one or more sensor 101, transmitter 102, communication link 103, receiver 104, and data processing terminal 105, where each receiver 104 is uniquely synchronized with a respective transmitter 102.

In one embodiment of the present invention, the sensor 101 is physically positioned on the body of a user whose glucose level is being monitored. The sensor 101 is configured to continuously sample the glucose level of the user and convert the sampled glucose level into a corresponding data signal for transmission by the transmitter 102. In one embodiment, the transmitter 102 is mounted on the sensor 101 so that both devices are positioned on the user's body. The transmitter 102 performs data processing such as filtering and encoding on data signals, each of which corresponds to a sampled glucose level of the user, for transmission to the receiver 104 via the communication link 103.

In one embodiment, the continuous glucose monitoring system 100 is configured as a one-way RF communication path from the transmitter 102 to the receiver 104. In such embodiment, the transmitter 102 transmits the sampled data signals received from the sensor 101 without acknowledgement from the receiver 104 that the transmitted sampled data signals have been received. For example, the transmitter 102 may be configured to transmit the encoded sampled data signals at a fixed rate (e.g., at one minute intervals) after the completion of the initial power on procedure. Likewise, the receiver 104 may be configured to detect such transmitted encoded sampled data signals at predetermined time intervals.

Additionally, in one aspect, the receiver 104 may include two sections. The first section is an analog interface section that is configured to communicate with the transmitter 102 via the communication link 103. In one embodiment, the analog interface section may include an RF receiver and an antenna for receiving and amplifying the data signals from the transmitter 102, which are thereafter, demodulated with a local oscillator and filtered through a band-pass filter. The second section of the receiver 104 is a data processing section which is configured to process the data signals received from the transmitter 102 such as by performing data decoding, error detection and correction, data clock generation, and data bit recovery.

In operation, upon completing the power-on procedure, the receiver 104 is configured to detect the presence of the transmitter 102 within its range based on, for example, the strength of the detected data signals received from the transmitter 102 or a predetermined transmitter identification information. Upon successful synchronization with the corresponding transmitter 102, the receiver 104 is configured to begin receiving from the transmitter 102 data signals corresponding to the user's detected glucose level. More specifically, the receiver 104 in one embodiment is configured to perform synchronized time hopping with the corresponding synchronized transmitter 102 via the communication link 103 to obtain the user's detected glucose level.

Referring again to FIG. 1, the data processing terminal 105 may include a personal computer, a portable computer such as a laptop or a handheld device (e.g., personal digital assistants (PDAs)), and the like, each of which may be configured for data communication with the receiver via a wired or a wireless connection. Additionally, the data processing terminal 105 may further be connected to a data network (not shown) for storing, retrieving and updating data corresponding to the detected glucose level of the user.

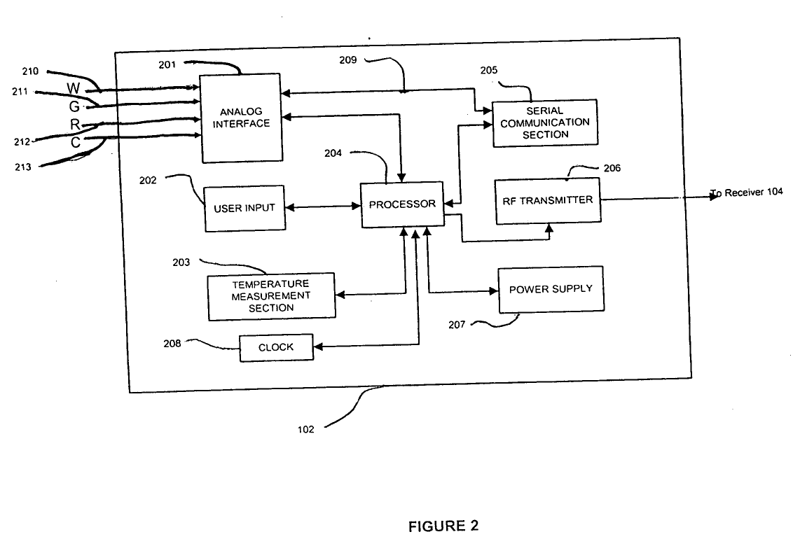

FIG. 2 is a block diagram of the transmitter of the data monitoring and detection system shown in FIG. 1 in accordance with one embodiment of the present invention. Referring to the Figure, the transmitter 102 in one embodiment includes an analog interface 201 configured to communicate with the sensor 101 (FIG. 1), a user input 202, and a temperature detection section 203, each of which is operatively coupled to a transmitter processor 204 such as a central processing unit (CPU). As can be seen from FIG. 2, there are provided four contacts, three of which are electrodes—work electrode (W) 210, guard contact (G) 211, reference electrode (R) 212, and counter electrode (C) 213, each operatively coupled to the analog interface 201 of the transmitter 102 for connection to the sensor unit 201 (FIG. 1). In one embodiment, each of the work electrode (W) 210, guard contact (G) 211, reference electrode (R) 212, and counter electrode (C) 213 may be made using a conductive material that is either printed or etched, for example, such as carbon which may be printed, or metal foil (e.g., gold) which may be etched.

Further shown in FIG. 2 are a transmitter serial communication section 205 and an RF transmitter 206, each of which is also operatively coupled to the transmitter processor 204. Moreover, a power supply 207 such as a battery is also provided in the transmitter 102 to provide the necessary power for the transmitter 102. Additionally, as can be seen from the Figure, clock 208 is provided to, among others, supply real time information to the transmitter processor 204.

In one embodiment, a unidirectional input path is established from the sensor 101 (FIG. 1) and/or manufacturing and testing equipment to the analog interface 201 of the transmitter 102, while a unidirectional output is established from the output of the RF transmitter 206 of the transmitter 102 for transmission to the receiver 104. In this manner, a data path is shown in FIG. 2 between the aforementioned unidirectional input and output via a dedicated link 209 from the analog interface 201 to serial communication section 205, thereafter to the processor 204, and then to the RF transmitter 206. As such, in one embodiment, via the data path described above, the transmitter 102 is configured to transmit to the receiver 104 (FIG. 1), via the communication link 103 (FIG. 1), processed and encoded data signals received from the sensor 101 (FIG. 1). Additionally, the unidirectional communication data path between the analog interface 201 and the RF transmitter 206 discussed above allows for the configuration of the transmitter 102 for operation upon completion of the manufacturing process as well as for direct communication for diagnostic and testing purposes.

As discussed above, the transmitter processor 204 is configured to transmit control signals to the various sections of the transmitter 102 during the operation of the transmitter 102. In one embodiment, the transmitter processor 204 also includes a memory (not shown) for storing data such as the identification information for the transmitter 102, as well as the data signals received from the sensor 101. The stored information may be retrieved and processed for transmission to the receiver 104 under the control of the transmitter processor 204. Furthermore, the power supply 207 may include a commercially available battery.

The transmitter 102 is also configured such that the power supply section 207 is capable of providing power to the transmitter for a minimum of three months of continuous operation after having been stored for 18 months in a low-power (non-operating) mode. In one embodiment, this may be achieved by the transmitter processor 204 operating in low power modes in the non-operating state, for example, drawing no more than approximately 1 μA of current. Indeed, in one embodiment, the final step during the manufacturing process of the transmitter 102 may place the transmitter 102 in the lower power, non-operating state (i.e., post-manufacture sleep mode). In this manner, the shelf life of the transmitter 102 may be significantly improved.

Referring yet again to FIG. 2, the temperature detection section 203 of the transmitter 102 is configured to monitor the temperature of the skin near the sensor insertion site. The temperature reading is used to adjust the glucose readings obtained from the analog interface 201. The RF transmitter 206 of the transmitter 102 may be configured for operation in the frequency band of 315 MHz to 322 MHz, for example, in the United States. Further, in one embodiment, the RF transmitter 206 is configured to modulate the carrier frequency by performing Frequency Shift Keying and Manchester encoding. In one embodiment, the data transmission rate is 19,200 symbols per second, with a minimum transmission range for communication with the receiver 104.

Additional detailed description of the continuous glucose monitoring system, its various components including the functional descriptions of the transmitter are provided in application Ser. No. 09/753,746 filed on Jan. 2, 2001 entitled “Analyte Monitoring Device and Methods of Use”, and in application Ser. No. 10/745,878 filed Dec. 26, 2003 entitled “Continuous Glucose Monitoring System and Methods of Use”, each assigned to the Assignee of the present application, and the disclosures of each of which are incorporated herein by reference for all purposes.

FIG. 3 illustrates the front end section of the analog interface of the transmitter in accordance with one embodiment of the present invention. Referring to the Figure, the front end section of the analog interface 201 includes a current to voltage circuit 301 which is configured to operatively couple to the work electrode 210 and the guard contact 211, and a counter-reference servo circuit 302 which is configured to operatively couple to the reference electrode 212 and the counter electrode 213.

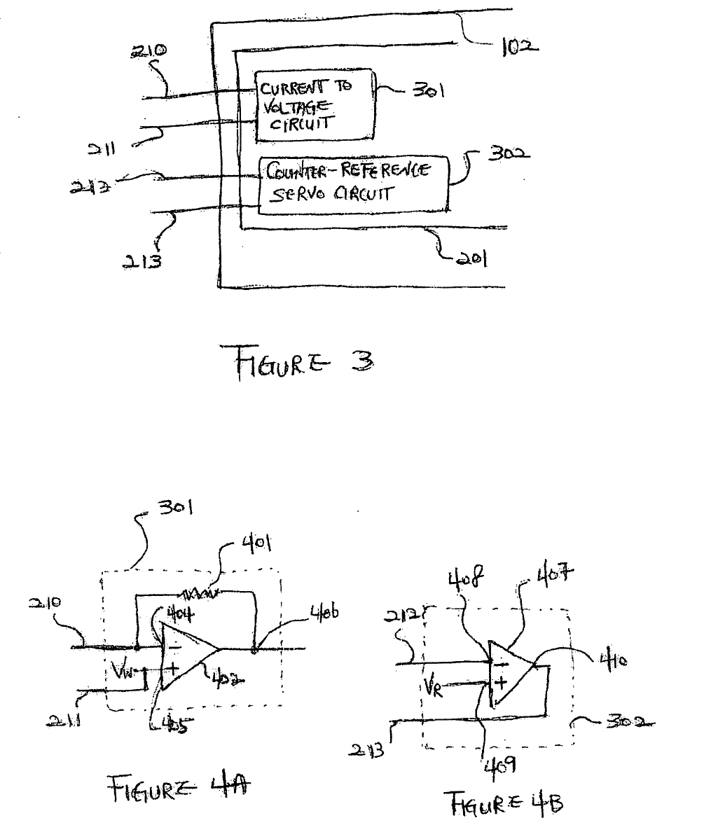

FIGS. 4A-4B illustrate detailed illustrations of the current to voltage circuit and the counter-reference servo circuit, respectively, of the analog interface shown in FIG. 3 in accordance with one embodiment of the present invention. Referring to FIG. 4A, the current to voltage circuit 301 (FIG. 3) in one embodiment includes an operational amplifier 402 having a non-inverting input terminal 405, and an inverting input terminal 404. Also shown in the Figure is a resistor 401 operatively coupled to the inverting input terminal 404 of the operational amplifier 402, and an output terminal 406.

Referring again to FIG. 4A, the work electrode 210 is operatively coupled to the inverting input terminal 404 of the operational amplifier 402, while the guard contact 211 is operatively coupled to the non-inverting input terminal 405 of the operational amplifier 402. It can be further seen that the work voltage source Vw is provided to the non-inverting terminal 405 of the operational amplifier 402. In this manner, in accordance with one embodiment of the present invention, a separate contact, the guard contact 211 is operatively coupled to the analog interface 201 (FIG. 2) of the transmitter 102 (FIG. 2). The guard contact 211 as discussed in further detail below is provided at a substantially equipotential to the work electrode 210 such that any current leakage path to the work electrode 210 (from either the reference electrode 212 or the counter electrode 213, for example) is protected by the guard contact 211 by virtue of maintaining the guard contact at substantially the same potential as the work electrode 210.

Referring now to FIG. 4B, the counter-reference servo unit 302 in accordance with one embodiment includes an operational amplifier 407 having an inverting input terminal 408 and a non-inverting input terminal 409, as well as an output terminal 410. In one embodiment, the reference electrode 212 is operatively coupled to the inverting input terminal 408, while the counter electrode 213 is operatively coupled to the output terminal 410 of the operational amplifier 407 in the counter-reference servo unit 302. It can also be seen from FIG. 4B that a reference voltage source Vr is provided to the non-inverting input terminal 409 of the operational amplifier 407 in the counter-reference servo unit 302.

Referring back to FIGS. 3 and 4A-4B, in accordance with one embodiment of the present invention, the current to voltage circuit 301 and the counter-reference servo unit 302 are operatively coupled to the remaining sections of the analog interface 201 of the transmitter 102, and configured to convert the detected glucose level at the sensor unit 101 (FIG. 1) into an analog signal for further processing in the transmitter unit 102. It should also be noted that, in the manner described, the Poise voltage (for example, at a value of 40 mV) may be determined based on the difference between the voltage signal level of the work voltage source Vw at the non-inverting input terminal 405 of the operational amplifier 402 in the current to voltage circuit 301, and the voltage signal level of the reference voltage source Vr at the non-inverting input terminal 409 of the operational amplifier 407 in the counter-reference servo unit 302.

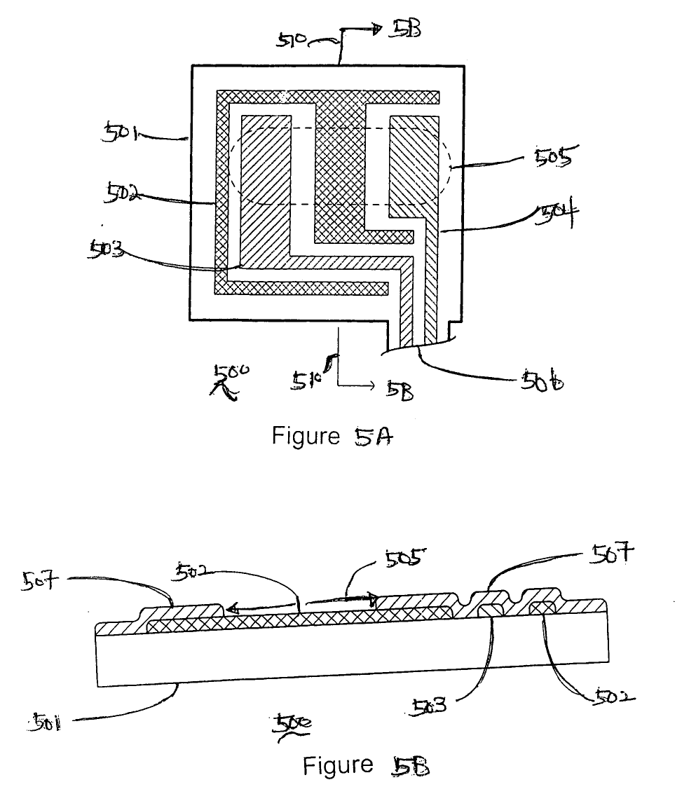

FIGS. 5A-5B illustrate a top view and a side view of the two electrode sensor with guard trace in accordance with one embodiment of the present invention. Referring to FIG. 5A, the two electrode sensor 500 includes a base material 501 such as Melinex (which is a polyester film similar to Mylar), and provided thereon, a guard trace 502 which is configured to comprise the guard contact 211 (FIG. 2), and which, as can be seen from FIG. 5A, is provided substantially entirely around the work trace 503. The work trace 503 as shown is configured to form the work electrode 210 (FIG. 2) and which is operatively coupled to the analog interface 201 of the transmitter 102 (FIG. 1). It can be further seen from FIG. 5A that the work trace 503 extends beyond the “flag” portion of the two electrode sensor 500 to the “tail” section for providing an electrode for the sensor unit 101 (FIG. 1).

Referring back to FIG. 5A, also shown is the counter/reference trace 504 which is provided on the base material 501 to form the counter electrode 213 (FIG. 2). It can also be seen that the counter trace 504 is substantially configured to extend beyond the “flag” portion of the sensor 500, similar to the work trace 503, and extend to the “tail” section 506 of providing an electrode for the sensor unit 101 (FIG. 1). In one embodiment, the “tail” section 506 is configured to provide active sensor/detection area. Moreover, a dielectric window 505 is provided to expose a predetermined area of the work trace 503 and the counter/reference trace 504 for providing contact areas with the electronic assembly of the analog interface 201 in the transmitter 102.

The area outside the dielectric window 505 may in one embodiment by coated with a protective (e.g., insulating) layer in which case, the guard trace 502 as shown may not need to extend much further beyond the dielectric window 505. In this case, the area around the guard trace 502 may be configured such that substantially all possible sensor contact positions (including valid and invalid positions) do not allow a conductive path from the work electrode 210 (FIG. 2) (shown here by the work trace 503) to any of the other electrodes that does not include a conductive path to the guard electrode 211 (FIG. 2) illustrated in FIG. 5A by the guard trace 502. Moreover, in accordance with one embodiment of the present invention, the guard trace 502 may not be extended into the “tail” section 506 of the two electrode sensor 500 so as to limit the width of the “tail” section 506 which comes into contact with the person's body, for example, and through which, the glucose level is detected and monitored.

Referring back, FIG. 5B illustrates the side view of the two electrode sensor 500 shown by the arrows 510 in FIG. 5A. More specifically, FIG. 5B illustrates the two electrode sensor 500 viewed in the direction of the arrows 510 and bisected therealong, and shows the base material 501, as well as the dielectric layer 507 (not shown in FIG. 5A) and the dielectric window 505 formed thereon. It can be further seen from FIG. 5B that the guard trace 502, as well as the work trace 503 are layered at their respective locations over the base material 501, and on top of which, is provided the dielectric layer 507. The portion of the guard trace 502 which does not have the dielectric layer 507 substantially corresponds to the section of the guard trace 502 shown in FIG. 5A which is within the dielectric window 505.

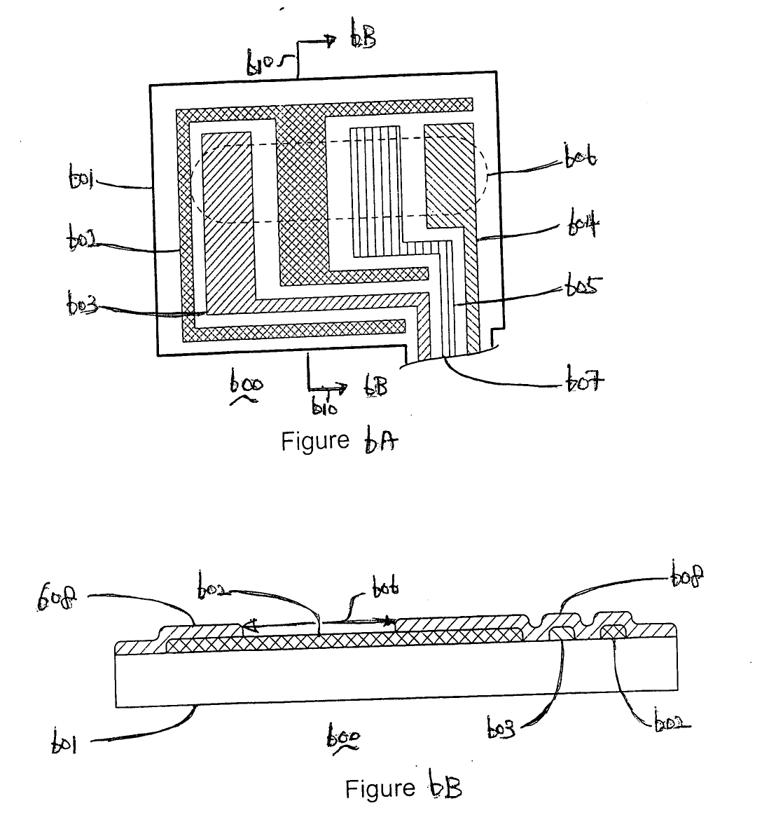

FIGS. 6A-6B illustrate a top view and a side view of the three electrode sensor with guard trace in accordance with one embodiment of the present invention. Referring to FIG. 6A, it can be seen that in this embodiment, a reference trace 605 is provided between the guard trace 602 and the counter trace 604. Similar to the two electrode sensor 500 embodiment shown in FIGS. 5A-5B, the dielectric window 606 shown in FIG. 6A provides a contact area to provide contacts with the electronic assembly of the analog interface 201 in the transmitter 102. As can be further seen from FIG. 6A, the guard trace 602, the work trace 603, the counter trace 604 and the reference trace 605, are each provided over the base material 601, and further, each of the work trace 603, the counter trace 604 and the reference trace 605 are provided so as to extend beyond the “flag” portion of the three electrode sensor 600 to the tail section 607 to provide the sensor electrodes to the person's skin (for example), for measuring the person's glucose level. Moreover, similar to the embodiment shown in FIGS. 5A-5B, the guard trace 602 is provided substantially to surround the work trace 603 over the entire three electrode sensor 600.

FIG. 6B illustrates a side view of the three electrode sensor 600 with guard trace 602 in accordance with one embodiment of the present invention. As can be seen, the embodiment shown in FIG. 6B is substantially similar to that shown in FIG. 5B as the side profile perspective of FIG. 6B which is viewed from the arrows 610 shown in FIG. 6A does not include the reference trace 605. Referring to FIG. 6B, it can be seen that the electrode trace layer for each of the guard trace 602, the work trace 603, the counter trace 604, and the reference trace 605 is provided on the base material 601, and further, that the dielectric layer 608 is provided on top of the electrode trace layer and the base material 601. Also, the dielectric window 606 which exposes a portion of the guard trace layer 602 is additionally shown in FIG. 6B.

In the manner described above, in accordance with one embodiment of the present invention, there is provided a sensor including a work electrode disposed on a base material, a reference electrode disposed on the base material, and a guard electrode disposed on the base material, wherein the guard electrode is disposed substantially around the work electrode.

In one embodiment, the base material may include one of Melinex or Mylar, or any other flexible biocompatible material. The guard electrode may be configured to be maintained substantially at equipotential to the work electrode. The guard electrode may be configured to protect a current leakage path to the work electrode.

In one embodiment, the guard electrode may be disposed substantially between the work electrode and the reference electrode. The sensor in another embodiment may include a counter electrode disposed on the base material. Also, the sensor may include a dielectric window disposed on the base material to expose a portion of the work and reference electrodes for electrical contact.

The dielectric window may be configured to provide the electrical contact of the work and reference electrodes to a transmitter in a data communication system, where transmitter in one embodiment may include a blood glucose monitoring meter. Moreover, in a further embodiment, the data communication system may include a blood glucose monitoring system including a continuous blood glucose monitoring system. In an additional embodiment, a protective layer may be disposed over said dielectric window on said base material, where the protective layer may include an insulation layer.

A glucose monitoring system in accordance with another embodiment of the present invention includes a sensor for detecting a glucose level, the sensor including, a work electrode disposed on a base material, a reference electrode disposed on the base material, and a guard electrode disposed on the base material, wherein the guard electrode is disposed substantially around the work electrode, and a transmitter operatively coupled to the work electrode and the reference electrode of the sensor for receiving the detected glucose level, the transmitter further configured to transmit a signal corresponding to the detected glucose level.

The transmitter may be configured to transmit the signal wirelessly. More specifically, in one embodiment, the transmitter may be configured to transmit the signal using one of a RF transmission protocol, a IrDA transmission protocol, a Bluetooth transmission protocol, a Zigbee transmission protocol, an 802.11x transmission protocol, and an infrared transmission protocol.

In another embodiment, the monitoring system may include a receiver operatively coupled to the transmitter, where the receiver may be configured to receive the transmitted signal corresponding to the detected glucose level. Moreover, the receiver may be configured to receive the transmitted signal over a wireless network. The receiver may include a blood glucose monitor configured to generate an output signal based on the received transmitted signal, where the output signal generated may include one or more of an alphanumeric, a two-dimensional graphic, a three-dimensional graphic, and an auditory representation of a blood glucose level corresponding to the detected glucose level.

Further, the receiver may include a display section, and further, wherein the generated output signal is displayed on the display section of the receiver, where the display section may include one of a Liquid Crystal Display, and a plasma display. Also, the generated output signal may be displayed in a graphical representation on the display section.

In a further embodiment, the sensor may be configured to detect a predetermined number of glucose levels over a predefined time period, and further, where the transmitter may be further configured to transmit the predetermined number of glucose levels substantially in real time relative to the corresponding detection by the sensor over the predefined time period.

Additionally, the monitoring system may include a receiver configured to receive the predetermined number of glucose levels over the predefined time period from the transmitter. Also, the receiver may be configured to receive the predetermined number of glucose levels over a wireless data network. The receiver may be further configured to generate one or more signals corresponding to each of the predetermined number of glucose levels received from the transmitter. Moreover, the receiver may be further configured to display the generated one or more signals substantially in real time relative to the reception of the corresponding glucose levels from the transmitter.

In a further embodiment, the guard electrode of the sensor may be configured to be maintained substantially at equipotential to the work electrode. The guard electrode may be configured to protect a current leakage path to the work electrode. The guard electrode may be disposed substantially between the work electrode and the reference electrode. The monitoring system in a further embodiment may include a counter electrode disposed on the base material. The monitoring system of one embodiment of the present invention may include a dielectric window disposed on the base material so as to expose a portion of the work and reference electrodes for electrical contact. Also, the dielectric window may be configured to provide the electrical contact of the work and reference electrodes to the transmitter. Additionally, a protective layer may be disposed over the dielectric window on the base material, and the protective layer may in one embodiment include an insulation layer.

The glucose monitoring system may in a further embodiment, include an insulin administration unit for administering an insulin dose based on the detected glucose level. The insulin administration unit may in one embodiment include an insulin pump configured to be in data communication with the transmitter. Furthermore, the insulin pump may be configured to include a receiver configured to receive the signal from the transmitter over a wireless data connection.

A method of providing a sensor for use in glucose monitoring system in accordance with yet another embodiment of the present invention includes the steps of disposing a work electrode on a base material, disposing a reference electrode on the base material, and disposing a guard electrode on the base material, wherein the guard electrode is disposed substantially around the work electrode. The method may further include the step of maintaining the guard electrode substantially at equipotential to the work electrode.

In one embodiment, the guard electrode may be disposed substantially between the work electrode and the reference electrode. The method may further include the step of disposing a counter electrode on the base material. The method may further include the step of disposing a dielectric window on the base material to expose a portion of the work and reference electrodes for electrical contact. Moreover, the method may additionally include the step of disposing a protective layer over the dielectric window on the base material, where the protective layer may include an insulation layer.

In the manner described above, in accordance with the various embodiments of the present invention, a separate guard contact or trace may be provided in a multiple electrode sensor configuration in portable electronic devices such as in continuous glucose monitoring systems, and which is maintained at a substantially equipotential to the work electrode, and provided substantially to physically encompass the work electrode, for example, so that any current leakage path to the work electrode from any of the other electrodes in the sensor configuration, is protected by the guard contact. Accordingly, potential error in the detected signals in the continuous glucose monitoring systems due to leakage current in the sensor of such systems may be minimized.

Indeed, as discussed above, a guard contact may be disposed at equipotential to the work electrode to substantially intercept all leakage currents by the guard contact such that the work electrode is unaffected even when foreign matter is present. In one embodiment, the guard contact may be provided between the work electrode and the reference electrode in a three-electrode sensor configuration, or alternatively, between the work electrode and counter/reference electrode in a two-electrode sensor configuration.

In a further embodiment, the guard trace may be used to surround the work electrode and associated traces to reduce leakage to the greatest possible extent for a given sensor configuration. Indeed, the guard trace may be extended from the system electronics through the contacts to the sensor to eliminate leakage currents resulting from contamination on the sensor. The extended guard contact and associated guard traces on the sensor in accordance with one embodiment may be configured to substantially minimize the potential for leakage current to the work electrode in sensor configurations so as to substantially eliminate potential adverse results such as erroneous data reading.

Additionally, within the scope of the present invention, the sensor configuration may include other insulating layers and electrode trace configurations. For example, in one aspect of the present invention, in a three electrode sensor configuration discussed above, an additional insulating layer may be provided between the base material and any of the reference electrode and the counter electrode. In a further embodiment, the work, reference and counter electrodes may be printed in a superimposed manner with interposed dielectric layers therebetween.

In this manner, in accordance with the various embodiments of the present invention, potential error in the detected signals in data communication systems such as in continuous glucose monitoring systems due to leakage current in the signal sensor configuration may be minimized.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims and their equivalents be covered thereby.

1. A sensor, comprising:
a work electrode disposed on a base material;
a reference electrode disposed on said base material; and
a guard electrode disposed on said base material, wherein the guard electrode is disposed substantially around said work electrode;
wherein at least a portion of one of the work or reference electrodes is disposed over at least a portion of the other one of the work or reference electrodes such that the at least portion of the other one of the work or reference electrodes is disposed between the at least the portion of the one of the work or reference electrodes and the base material.

2. The sensor of claim 1 wherein the base material includes one of Melinex or Mylar.

3. The sensor of claim 1 wherein the guard electrode is configured to be maintained substantially at equipotential to the work electrode.

4. The sensor of claim 1 wherein the guard electrode is configured to protect a current leakage path to said work electrode.

5. The sensor of claim 1 wherein the guard electrode is disposed substantially between said work electrode and said reference electrode.

6. The sensor of claim 1 further including a counter electrode disposed on said base material.

7. (canceled)

8. (canceled)

9. (canceled)

10. (canceled)

11. The sensor of claim 1 further including a protective layer disposed on said base material.

12. The sensor of claim 11 wherein the protective layer includes a window to expose a portion of one of the work or reference electrodes.

13. A glucose monitoring system, comprising:
a sensor for detecting a glucose level, the sensor including:
a work electrode disposed on a base material;
a reference electrode disposed on said base material;
a guard electrode disposed on said base material, wherein the guard electrode is disposed substantially around said work electrode;
wherein at least a portion of one of the work or reference electrodes is disposed over at least a portion of the other one of the work or reference electrodes such that the at least the portion of the other one of the work or reference electrodes is disposed between the at least the portion of the one of the work or reference electrodes and the base material; and
a transmitter operatively coupled to the work electrode and the reference electrode of the sensor for receiving said detected glucose level, said transmitter further configured to transmit a signal corresponding to said detected glucose level.

14. The glucose monitoring system of claim 13 wherein the transmitter is configured to transmit said signal wirelessly.

15. The glucose monitoring system of claim 14 wherein said transmitter is configured to transmit said signal using one of a RF transmission protocol, a IrDA transmission protocol, a Bluetooth transmission protocol, a Zigbee transmission protocol, an 802.11x transmission protocol, or an infrared transmission protocol.

16. The glucose monitoring system of claim 13 further including a receiver operatively coupled to said transmitter, said receiver configured to receive said transmitted signal corresponding to said detected glucose level.

17. The glucose monitoring system of claim 16 wherein said receiver is configured to receive said transmitted signal over a wireless network.

18. The glucose monitoring system of claim 16 wherein said receiver includes a blood glucose monitor configured to generate an output signal based on said received transmitted signal.

19. The glucose monitoring system of claim 18 wherein said output signal generated includes one or more of an alphanumeric, a two-dimensional graphic, a three-dimensional graphic, or an auditory representation of a glucose level corresponding to said detected glucose level.

20. The glucose monitoring system of claim 18 wherein said receiver includes a display section, and further, wherein said generated output signal is displayed on said display section of said receiver.

21. The glucose monitoring system of claim 20 wherein said display section includes one of a Liquid Crystal Display, or a plasma display.

22. The glucose monitoring system of claim 20 wherein the generated output signal is displayed in a graphical representation on said display section.

23. The glucose monitoring system of claim 13 wherein said sensor is configured to detect a predetermined number of glucose levels over a predefined time period, and further, wherein said transmitter is further configured to transmit said predetermined number of glucose levels substantially in real time relative to the corresponding detection by the sensor over the predefined time period.

24. The glucose monitoring system of claim 23 further including a receiver configured to receive said predetermined number of glucose levels over said predefined time period from said transmitter.

25. The glucose monitoring system of claim 24 wherein said receiver is configured to receive said predetermined number of glucose levels over a wireless data network.

26. The glucose monitoring system of claim 23 wherein said receiver is further configured to generate one or more signals corresponding to each of said predetermined number of glucose levels received from said transmitter.

27. The glucose monitoring system of claim 23 wherein said receiver is further configured to display said generated one or more signals substantially in real time relative to the reception of the corresponding glucose levels from said transmitter.

28. The glucose monitoring system of claim 13 wherein the guard electrode of the sensor is maintained substantially at equipotential to the work electrode.

29. The glucose monitoring system of claim 13 wherein the guard electrode is configured to protect a current leakage path to said work electrode.

30. The glucose monitoring system of claim 13 wherein the guard electrode is disposed substantially between said work electrode and said reference electrode.

31. The glucose monitoring system of claim 13 further including a counter electrode disposed on said base material.

32. (canceled)

33. (canceled)

34. The glucose monitoring system of claim 13 further including a protective layer disposed on said base material.

35. The glucose monitoring system of claim 34 wherein the protective layer includes a window to expose a portion of one of the work or reference electrodes.

36. The glucose monitoring system of claim 13 further including an insulin administration unit for administering an insulin dose based on said detected glucose level.

37. The glucose monitoring system of claim 36 wherein said insulin administration unit includes an insulin pump configured to be in data communication with said transmitter.

38. The glucose monitoring system of claim 37 wherein said insulin pump includes a receiver configured to receive the signal from said transmitter over a wireless data connection.

39. A method of providing a sensor for use in glucose monitoring system, comprising the steps of:
disposing a work electrode on a base material;
disposing a reference electrode on said base material;
disposing a guard electrode on said base material, wherein the guard electrode is disposed substantially around said work electrode; and
wherein at least a portion of one of the work or reference electrodes is disposed over at least a portion of the other one of the work or reference electrodes such that the at least the portion of the other one of the work or reference electrodes is disposed between the at least the portion of the one of the work or reference electrodes and the base material.

40. The method of claim 39 further including the step of maintaining the guard electrode substantially at equipotential to the work electrode.

41. The method of claim 39 wherein the step of disposing said guard electrode including the step of disposing said guard electrode substantially between said work electrode and said reference electrode.

42. The method of claim 39 further including the step of disposing a counter electrode on said base material.

43. (canceled)

44. The method of claim 39 further including the step of disposing a protective layer over said dielectric window on said base material.

45. The method of claim 45 wherein the protective layer includes a window to expose a portion of one of the work or reference electrodes.

46. The sensor of claim 1 further including a dielectric layer disposed between the base material and the one or more work or reference electrodes.

47. The glucose monitoring system of claim 13 wherein the sensor further includes a dielectric layer disposed between the base material and the one or more work or reference electrodes. 48. The method of claim 39 further including disposing a dielectric layer between the base material and the one or more work or reference electrodes.
